(19) United States
(12) Patent Application Publication
(10) Pub. No.: US 2021/0138246 A1
(43) Pub. Date: May 13, 2021

(54) SYSTEM AND METHOD FOR SEIZURE DETECTION AND AUTOMATED THALAMIC STIMULATION

(71) Applicant: Ivan Osorio, Leawood, KS (US)

(72) Inventors: Ivan Osorio, Leawood, KS (US); Richard Wennberg, Toronto (CA)

(21) Appl. No.: 17/150,908

(22) Filed: Jan. 15, 2021

Related U.S. Application Data
(63) Continuation-in-part of application No. 15/528,984, filed on May 23, 2017.
(60) Provisional application No. 62/083,511, filed on Nov. 24, 2014.

(51) Int. Cl. A61N 1/36; A61N 1/05

(52) U.S. Cl. CPC A61N 1/36064; A61N 1/36139; A61N 1/0531; A61N 1/0534

(57) ABSTRACT

A thalamic medical device system and method enables automated detection of epileptic seizures and optionally providing contingent thalamic stimulation triggered by automated seizure detection. The method includes detecting electrical signals in a frequency band of 8-42 Hz from an anterior thalamic nucleus or a centro-median thalamic nucleus in a first hemisphere and/or a second hemisphere of a brain with one or more electrode assemblies surgically implanted at least partially within an anterior thalamic nuclei or a centro-median thalamic nuclei, and outside of a subthalamic nuclei of the patient. The method also includes transmitting the detected electrical signals to a medical device, analyzing them using a seizure detection algorithm, and delivering electrical stimulation to the anterior thalamic nuclei or centro-median thalamic nuclei in the first hemisphere and/or the second hemisphere via the electrode assemblies in response to a seizure detected by the medical device.

### RELATED APPLICATION

This application is a Continuation-In-Part of U.S. patent application Ser. No. 15/528,984, filed May 23, 2017 (published as US20170265764), which is the U.S. National Stage of International Application No. PCT/US2015/062550, filed Nov. 24, 2015, which claims the benefit of U.S. Provisional Application No. 62/083,511, filed Nov. 24, 2014, the contents of each of which are herein incorporated by reference in their entireties.

### TECHNICAL FIELD

This invention relates to medical device systems and methods related to automated detection from the anterior nucleus or the centro-median nucleus of the thalamus of seizures originating outside these nuclei and contingent delivery of therapy via the same nuclei.

### BACKGROUND

Approximately 1% of the world population has epilepsy and approximately 36% of those with epilepsy are pharmaco-resistant by not responding well or at all to drugs intended to reduce seizures. For this large population of epileptics who cannot improve their condition through pharmaceutical drugs, they frequently suffer a wide array of chronic and acute medical and social needs (e.g., uncontrolled seizures, unemployment, inability to drive vehicles, depression, and even death).

Various forms of neurostimulation have been used to treat epileptic seizures, such as deep brain stimulation (DBS) and vagus nerve stimulation (VNS). These forms of neurostimulation have succeeded in treating numerous partial and generalized seizure types. Most DBS devices deliver electrical stimulation to the subthalamic nuclei, which is located below the thalamus. A need exists to explore additional neural structures aside from the: a) cortex, mesial temporal structures and certain subcortical but extra-thalamic structures (e.g., subthalamic nuclei, Forel's fields) to be used to detect epileptic seizures and deliver neurostimulation; b) simpler, easier to implant, smaller, safer and less costly methods and systems of automatically detecting and abating epileptic seizures for patients with more than one epileptogenic brain region and/or difficult to localize sites of epileptogenesis, and c) methods, apparati and systems that are more ergonomic and acceptable to patients than the current state-of the-art.

### SUMMARY

A need exists for detecting seizures and stimulating from within the thalamus by using contingent therapy delivery. Accordingly, in an aspect, a medical device system for automated detection of epileptic seizures can comprise: a first electrode assembly implanted into an anterior thalamic nucleus or a centro-median thalamic nucleus in a first hemisphere of a brain of a patient; a second electrode assembly implanted into an anterior thalamic nucleus or a centro-median thalamic nucleus in a second hemisphere of the patient's brain; and a medical device operatively connected to one or both of the first and second electrode assemblies, configured to detect electrical signals and analyze detected electrical signals using a seizure detection algorithm.

In some aspects, the first and second electrode assemblies are positioned outside of a subthalamic nuclei of the patient. In certain aspects, the medical device is configured to deliver electrical stimulation to the anterior thalamic nuclei or centro-median thalamic nuclei via one or both of the first and second electrode assemblies when a seizure is detected.

In an aspect, a medical device system for contingent thalamic stimulation triggered by automated seizure detection can comprise: an electrode assembly having at least a first electrode and a second electrode implanted into an anterior thalamic nuclei or a centro-median thalamic nuclei of a patient; a medical device operatively connected to the electrode assembly, configured to detect electrical signals and analyze detected electrical signals using a seizure detection algorithm, and configured to deliver electrical stimulation to the anterior thalamic nuclei or centro-median thalamic nuclei via the electrode assembly when a seizure is detected.

In certain aspects, the electrode assembly further comprises a third electrode positioned outside of and proximate to the anterior thalamic nuclei or the centro-median thalamic nuclei. In some aspects, the medical device is configured to reduce or disable the electrical stimulation delivered by at least one of the first, second, or third electrodes. In various aspects, the medical device is configured to disable electrical stimulation delivered by the third electrode. In some aspects, the medical device is configured to detect electrical signals via the third electrode, while electrical stimulation is disabled, and analyze detected electrical signals using the seizure detection algorithm. In some aspects, the third electrode is disabled based on detected electrical signals from the third electrode or a therapeutic response to prior electrical stimulation provided by the third electrode. In certain aspects, the third electrode is disabled based on medical imaging determining the third electrode is positioned outside of the anterior thalamic nuclei or the centro-median thalamic nuclei.

In some aspects, the medical device system further comprises a fourth electrode connected to the electrode assembly, wherein any two of the first through fourth electrodes are configured to detect electrical signals and to deliver electrical stimulation to the anterior thalamic nuclei or the centro-median thalamic nuclei.

In an aspect, a method for contingent thalamic stimulation triggered by automated seizure detection can comprise: detecting electrical signals from an electrode assembly positioned at least partially within an anterior thalamic nuclei or a centro-median thalamic nuclei of a patient; transmitting the detected electrical signals to a medical device; analyzing the detected electrical signals using a seizure detection algorithm; and delivering electrical stimulation to the anterior thalamic nuclei or centro-median thalamic nuclei via the electrode assembly when a seizure is detected by the medical device.

In some aspects, the electrode assembly is positioned outside of a subthalamic nuclei of the patient. In certain aspects, the electrode assembly comprises at least four electrodes having at least one pair of electrodes positioned within the anterior thalamic nuclei or centro-median thalamic nuclei. In various aspects, the method further comprises selectively detecting electrical signals from any pair of electrodes of the electrode assembly. In some aspects, the method further comprises selectively determining any pair of electrodes of the electrode assembly for delivering the electrical stimulation, the selective determination being based on at least one of: detected electrical signals; a therapeutic response to prior electrical stimulation; or medical imaging determining that at least one electrode is positioned outside of the anterior thalamic nuclei or the centro-median thalamic nuclei.

According to one aspect, a method for contingent thalamic stimulation triggered by automated seizure detection includes detecting electrical signals in a frequency band of 8-42 Hz from an anterior thalamic nucleus or a centro-median thalamic nucleus in a first hemisphere and/or a second hemisphere of a brain of a patient with one or more electrode assemblies surgically implanted at least partially within an anterior thalamic nuclei or a centro-median thalamic nuclei of the patient, and outside of a subthalamic nuclei of the patient. The method also includes transmitting the detected electrical signals to a medical device, analyzing the detected electrical signals using a seizure detection algorithm, and delivering electrical stimulation to the anterior thalamic nuclei or centro-median thalamic nuclei in the first hemisphere and/or the second hemisphere of the brain of the patient via the one or more electrode assemblies in response to a seizure detected by the medical device.

Particular embodiments may comprise one or more of the following features. Analyzing the detected electrical signals using the seizure detection algorithm may include determining whether a sudden increase in power of the electrical signals reaches a threshold value. The one or more electrode assemblies may include between one to four electrodes having at least one of the electrodes surgically implanted within the anterior thalamic nuclei or centro-median thalamic nuclei. The method may further include selectively detecting electrical signals from any of the electrodes of the one or more electrode assemblies. The method may further include selectively determining any of the electrodes of the one or more electrode assemblies for delivering the electrical stimulation. The selective determination may be based on at least one of: detected electrical signals and a therapeutic response to prior electrical stimulation. The method may further include detecting electrical signals from a cortical electrode array having a plurality of cortical electrodes. The cortical electrode array may be surgically implanted beneath the patient's skin and may be operatively connected to the medical device. The medical device may be configured to analyze electrical signals detected by the cortical electrodes using a cortical seizure detection algorithm.

According to another aspect of the disclosure, a method for contingent thalamic stimulation triggered by automated seizure detection includes detecting electrical signals in a frequency band of 8-42 Hz from an anterior thalamic nucleus or a centro-median thalamic nucleus in a first hemisphere and/or a second hemisphere of a brain of a patient with one or more electrode assemblies surgically implanted at least partially within an anterior thalamic nuclei of the patient while bypassing a subthalamic nuclei of the patient. The method also includes transmitting the detected electrical signals to a medical device, analyzing the detected electrical signals using a seizure detection algorithm, and delivering electrical stimulation to the anterior thalamic nuclei in the first hemisphere and/or the second hemisphere of the brain of the patient via the one or more electrode assemblies in response to a seizure detected by the medical device.

Particular embodiments may comprise one or more of the following features. The one or more electrode assemblies may include between one to four electrodes having at least one of the electrodes surgically implanted within the anterior thalamic nuclei. The method may further include selectively detecting electrical signals from any of the electrodes of the one or more electrode assemblies. The method may further include detecting electrical signals from a cortical electrode array having a plurality of cortical electrodes.

According to yet another aspect of the disclosure, a method for contingent thalamic stimulation triggered by automated seizure detection includes detecting electrical signals in a frequency band of 8-42 Hz from an anterior thalamic nucleus or a centro-median thalamic nucleus in a first hemisphere and/or a second hemisphere of a brain of a patient with one or more electrode assemblies surgically implanted at least partially within a centro-median thalamic nuclei of the patient, the one or more electrode assemblies also dodging a subthalamic nuclei of the patient. The method also includes transmitting the detected electrical signals to a medical device, analyzing the detected electrical signals using a seizure detection algorithm, and delivering electrical stimulation to the centro-median thalamic nuclei in the first hemisphere and/or the second hemisphere of the brain of the patient via the one or more electrode assemblies in response to a seizure detected by the medical device.

Particular embodiments may comprise one or more of the following features. The one or more electrode assemblies may include between one to four electrodes having at least one of the electrodes surgically implanted within the centro-median thalamic nuclei. The method may further include selectively detecting electrical signals from any of the electrodes of the one or more electrode assemblies. Analyzing the detected electrical signals using the seizure detection algorithm may include determining whether a sudden increase in power of the electrical signals reaches a threshold value.

According to still another aspect of the disclosure, a method for contingent thalamic stimulation triggered by automated seizure detection includes surgically implanting one or more electrode assemblies into an anterior thalamic nucleus or a centro-median thalamic nucleus in a first hemisphere and/or a second hemisphere of a brain of a patient for the automated detection and contingent electrical treatment of seizures. The method also includes determining post-operatively, using imaging tests, a location of the electrode assemblies in relation to the anterior thalamic nucleus or the centro-median nucleus of a patient, as well as modifying at least one of a stimulation parameter, a distance between electrodes receiving electrical current, and a location of a positive current sink, if the electrode assemblies are not at least in the vicinity the intended target structure. Lastly, the method includes delivering an adjusted electrical stimulation to the vicinity of the anterior thalamic nuclei or centro-median thalamic nuclei in the first hemisphere and/or the second hemisphere of the brain of the patient via the one or more electrode assemblies in response to a seizure detected by the medical device. Particular embodiments may comprise one or more of the following features. The stimulation parameter modified may be one of the current intensity and the voltage.

Aspects and applications of the disclosure are described below with reference to the drawings and the detailed description. Unless specifically noted, the words and phrases in the specification and the claims should be given their plain, ordinary, and accustomed meaning to those of ordinary skill in the applicable arts. The inventors are fully aware that they can be their own lexicographer if desired. The inventors expressly elect, as their own lexicographer, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly comprise additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

### BRIEF DESCRIPTION OF THE DRAWINGS

FIG. 1 shows a non-limiting example of a thalamic medical device system for automated detection of epileptic seizures and neurostimulation.

FIG. 2 shows a non-limiting example of an electrode assembly implanted in the thalamus of a patient.

FIG. 3 shows a non-limiting example of a perspective exploded view of the thalamus where an electrode assembly has been implanted in the centro-median thalamic nucleus.

### DETAILED DESCRIPTION

This disclosure, its aspects and implementations, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

The words “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

In the following description, and for the purposes of explanation, numerous specific details are set forth in order to provide a thorough understanding of the various aspects of the disclosed embodiments. It will be understood, however, by those skilled in the relevant arts, that the present disclosed embodiments may be practiced without these specific details. In other instances, known structures and devices are shown or discussed more generally in order to avoid obscuring the disclosed embodiments. In many cases, a description of the operation is sufficient to enable one to implement the various forms of the disclosed embodiments, particularly when the operation is to be implemented in software. It should be noted that there are many different and alternative configurations, devices and technologies to which the disclosed embodiments may be applied. The full scope of the disclosed embodiments are not limited to the examples that are described below.

Both epileptic seizures and non-epileptic seizures have customarily been identified by observing the behavior of the patient having the seizure and through analysis of electrical brain signals. These neural oscillations or brain signals are measurable by detecting the potential difference (voltage) between two locations in the brain. Electroencephalography (EEG) can provide many channels of brain signal data, but scalp EEG signals have poor spatial resolution and contain significant muscle noise, resulting in low sensitivity and specificity when compared to brain signals detected from intra-cranial electrodes. Intra-cranial electrodes surgically implanted beneath the cranium (e.g., epidural, subdural, or depth electrodes) have significantly improved signal to noise ratios when compared to scalp EEG recordings of brain signals. Another possibility for electrode placement is beneath the scalp but above the outer skull table, which has several advantages over scalp EEG and several disadvantages over intra-cranial electrodes.

Implantable medical devices may deliver therapeutic electrical stimulation to the brain or other neural structures to treat patients suffering from epilepsy. As used herein, “stimulation,” “neurostimulation,” “electrical stimulation,” “stimulation signal,” “therapeutic signal,” or “neurostimulation signal” refers to an application of an electrical charge applied to an organ or a neural structure in the patient's body. The intent of applying neurostimulation is to prevent, stop, or minimize a seizure by creating a suppressing (e.g., blocking) or modulating effect to the targeted neural tissue. A medical device may deliver scheduled periodic stimulation (i.e., previously programmed) or contingent stimulation. Contingent stimulation (also referred to as “closed-loop,” “active,” or “feedback” neurostimulation) is neurostimulation applied in response to sensed information, such as brain signals detected by electrodes that are indicative of a seizure.

While seizure detection is universally performed through the analyses of electrical signals originating from the neocortex or from deeply located structures in the mesial temporal lobes, subcortical seizure detection can be accomplished using the disclosed system and methods of automated detection and quantification of seizures from the thalamus. Subcortical seizure detection and treatment have important advantages over the cortical or amygdalo-hippocampal since it requires insertions into the brain less hardware (e.g., electrodes) and less signal acquisition. Detecting and stimulating from within the thalamus by using contingent therapy delivery is particularly valuable for patients with multiple or difficult to localize epileptogenic regions. Thalamic electrical stimulation is a worthwhile therapeutic option for patients with pharmaco-resistant epilepsy. Contingent thalamic stimulation triggered by automated seizure detections may improve efficacy since it: a) would be delivered in close temporal proximity to electrographic onset, an early intervention that decreases the probability of spread and intensification or may actually abort the seizure; b) would permit quantification of seizure frequency, intensity and duration and thereby enable better, more informed stimulation parameter optimization. The disclosed medical device system may use the same electrode assembly for seizure detection and therapy delivery (e.g., stimulation), and provide the ability to detect seizures and deliver therapy in close temporal proximity to seizure onset.

FIG. 1 depicts a non-limiting example of a thalamic medical device system 100 for automated detection of epileptic seizures and optionally providing contingent thalamic stimulation triggered by automated seizure detection. The thalamic medical device system 100 includes a medical device 110 connected to an electrode assembly 120 using a lead 115. The electrode assembly 120 has at least two electrodes 130 (e.g., 2, 3, 4, 6, 10, etc.), where at least one electrode 130 is surgically implanted into an anterior thalamic nuclei (“ATN”) 162 or a centro-median thalamic nuclei (“CMN”) 164 of the brain 152 of a patient 150 (see FIGS. 2-3). medical device 110 includes a processor 112 and memory 113 configured to execute a seizure detection algorithm 111 that analyzes brain signals to detect or predict seizures in patient 150. Electrode assembly 120, electrodes 130, and lead 115 may be deep brain stimulation (“DBS”) electrodes configured to sense and/or stimulate neural structures below the cortex of brain 152. In certain embodiments, the thalamic medical device system 100 also includes a second medical device 210 connected to an electrode assembly 220 using a lead 215. This optional second medical device 210 operates identical or similar to medical device 110 and is configured to detect electrical brain signals from or deliver electrical stimulation to another neural structure (e.g., electrode assembly 120 is in the left ATN and electrode assembly 220 is in the right ATN). In alternative embodiments, both the electrode assembly 220 and lead 215 and the electrode assembly 220 and lead 215 are connected (not shown) to medical device 110, such that medical device 110 operates to control the seizure detection and (optional) neurostimulation at both electrode assemblies 120 and 220. Thus, thalamic medical device system 100 can detect seizures from and deliver electrical stimulation to multiple neural structures by using a single medical device 110 or also a second medical device 210.

FIGS. 2 and 3 depict the electrode assembly 120 surgically implanted in the brain 152 of patient 150. The disclosed thalamic medical device system 100 includes at least one electrode assembly 120 having at least one electrode 130 implanted within one of four neural structures: left ATN 162, left CMN 164, right ATN 162, or right CMN 164. In some embodiments, two electrode assemblies are bilaterally implanted either into: a) the anterior thalamic nuclei (ATN 162); b) the centro-median thalamic nuclei (CMN 164) (e.g., electrode assembly 120 in left ATN 162 and electrode assembly 220 in right ATN 162); c) the ATN and the ipsilateral CMN, or d) the ATN and the contralateral CMN. In FIG. 2, electrode assembly 120 has been surgically implanted (e.g., stereotactic implantation) so that at least one electrode 130 is positioned within the left ATN 162. For example, the ATN 162 in many patients has a height of approximately 6 mm and using an electrode assembly 120 having four electrodes 130 spanning a length of 10.5 mm, would result in at least one electrode 130 positioned outside of ATN 162 (e.g., where two or three of electrodes 130 are located within the ATN 162 and one or two of electrodes 130 are located in the dorso-median thalamic nucleus).

Implantation of electrode assembly 120 into the left or right ATN 162 or CMN 164 can take any one of many paths through brain 152 (e.g., implanted from anterior, posterior, superior, inferior, left, or right starting points surrounding brain 152), and is not required to be implanted from above as shown. A cavity 158 is created in the skull 156 to expose the dura mater covering the brain 152. A guide cover 122 may be affixed to the skull 156 in the cavity 158 to help the electrode assembly 120 remain in position after surgical implantation. The lead 115 is flexible and generally is implanted with extra length and one or more loops to allow the medical device 110 and lead 115 to move without adjusting the position of the implanted electrode assembly 120.

FIG. 3 shows a perspective exploded view of the thalamus 160 (only the left half is shown) where, in a non-limiting example, the electrode assembly 120 has been surgically implanted in the centro-median thalamic nucleus 164. In this example, electrode assembly 120 includes four electrodes 130, labeled as electrodes 131, 132, 133, and 134. A combination of any two of electrodes 131-134 may be used to detect electrical brain signals and/or deliver electrical stimulation. The pair of electrodes 130 selected from electrodes 131-134 may be an adjacent pair (e.g., electrodes 131/132, 132/133, or 133/134) or may be a non-adjacent pair (e.g., electrodes 131/133, 131/134, or 132/134). The same pair or pairs of electrodes 130 may be used when detecting seizures and during neurostimulation. Alternatively, one or more different pair(s) of electrodes 130 may be used when detecting seizures and during neurostimulation.

Of note, electrode 134 is not positioned within CMN 164 because CMN 164 is a relatively small neural structure compared to the electrode assembly 120 depicted in FIG. 3. When one or more electrodes 130 are not positioned within, or in the vicinity of, the targeted neural structure (e.g., ATN 162 or CMN 164), the sensing and/or stimulating operations from those electrode(s) 130 positioned outside of the target neural structure (e.g., electrode 134), such as the sampling time and/or frequency and the delivery of electrical current (for therapeutic purposes), may be reduced or disabled.

Precise placement of probes/electrodes into a designated subcortical structure of the human brain is difficult to accomplish, as ascertained by imaging studies (e.g., CT scans, etc.) performed after their surgical implantation. Commonly, the probe may be “off-target” by a few millimeters in the coronal, axial or sagittal plane. In clinical practice, this translates into decreased efficacy of an intended therapy (e.g., electrical stimulation thorough depth electrodes) or in the sensitivity and specificity of detection of certain events (e.g., seizures) from the intended brain structure. Re-implantation of the electrodes to improve placement not only increases the risk of serious complications and cost of care, but more importantly, does not guarantee precise placement.

According to various embodiments, a safer and more cost effective approach to improve target acquisition after implantation of electrodes of the system contemplated herein is to increase the size of the electrical field so as to better encompass the intended target structure. In some embodiments, this may be accomplished by increasing the intensity of the current delivered through the electrodes, while in other embodiments this may be accomplished by increasing the number of electrodes through which current is delivered. In still other embodiments, the size of the field may be modified by changing the distance between active electrodes (e.g. modifying distance by selecting different electrodes to activate, etc.) and/or adopting a “double mono-polar” configuration (e.g., negative depth electrodes to a positive case situated at a distance from them) through which current is being passed. A quantitative study of the volume of axonal tissue directly activated by electrical “bipolar” stimulation of the subthalamic nucleus with parameters (3 V; 0.1 ms; 150 Hz) showed axon activation as far as 4 mm from the electrode contact (McIntyre et al, 2004). Since for therapeutic purposes current intensity delivered to brain tissue is usually 5 V or higher, the spread of current will be greater than 4 mm from the electrode surface, thus facilitating stimulation of targets at located at a distance from the depth electrodes. In the context of the present description and the claims that follow, a depth electrode is considered to be in the vicinity of the intended target structure if its main axis is within 5 millimeters of the outermost margins of said target in the coronal, sagittal or axial planes.

Using the disclosed thalamic medical device system 100, thalamic electrical signals may be analyzed using seizure detection algorithm 111. In some embodiments, seizure detection algorithm 111 (SDA 111) estimates the relative increase in median power in the seizure content of the raw brain signals in a weighted frequency band (e.g., 8-42 Hz). Seizures are operationally defined as a sudden increase in power in the frequency band (e.g., 8-42 Hz), reaching a certain threshold value. The SDA 111 may be run on the subcortical thalamic brain signals detected by electrode assembly 120 using in differential (bipolar) channels created from adjacent electrodes 130 (e.g., four electrodes 131-134) or in referential montage (e.g., electrode 131 referred to an indifferent electrode (not shown)). If a simultaneously measured scalp EEG is compared to the measured brain signals from the electrode assembly 120, many seizures will evidence differences in onset times where the thalamic electrode assembly 120 detects seizure events before the brain signals reach the scalp EEG. Sometimes, a scalp EEG will not even measure evidence of a seizure event that is clearly shown using thalamic electrode assembly 120 in the thalamic medical device system 100. In a study of a 19-year-old man, for example, in 26 out of 28 seizures ictal activity was first detected in the right ATN approximately two seconds prior to clinical onset and 20-25 seconds prior to the first rhythmic ictal scalp changes were measured on a scalp EEG.

### Example

Ictal activity was recorded from the scalp (using scalp EEG) and thalamic nuclei (ATN 162 or CMN 164) in 3 subjects who had seizures during a 3-4 day recording period after DBS electrode implantation using one electrode assembly 120 or bilateral implantation of electrode assemblies 120 and 220. In the majority of seizures, ictal activity in the thalamic nuclei (ATN 162 or CMN 164) preceded electrographic onset as determined from the scalp EEG or clinical onset as determined from behavioral observations. Interictal epileptiform discharges were also recorded from the thalamus and in certain instances had no representation on the scalp EEG recording.

All three subjects suffered from medically intractable epilepsy and were part of an ongoing clinical trial designed to investigate the feasibility, safety and efficacy of periodic bilateral thalamic electrical stimulation. Deep brain stimulating (DBS) electrodes (model 3387, Medtronic, Minneapolis, USA) were surgically implanted bilaterally into the anterior thalamic nuclei (ATN 162; 7 patients) or into the centro-median thalamic nuclei (CMN 164; 2 patients). After stereotactic electrode assembly 120 implantation the thalamic electrodes were externalized through percutaneous extensions and subjects were transferred to the epilepsy monitoring unit where recordings were obtained from the thalamic electrode assembly 120 (impedances ˜1 Kohm) and from 21 scalp EEG electrodes (10-20 system) using 32 channel digital systems (Stellate, Montreal, Canada; XLTEK, Oakville, Canada; 0.5-70 Hz; 200 Hz digitization rate).

Four of nine patients had clinical seizures during the perioperative monitoring phase, which lasted 3-4 days. In one patient with drop attacks progressing to generalized tonic seizures, a subtle generalized electro-decremental pattern appeared prior to clinical onset, but no rhythmic ictal activity was recorded from the scalp EEG or thalamus electrode assemblies 120 and this patient was thus excluded from further analysis.

Subject 1: A 43-year-old man with left hemispheric, temporal neocortical, partial onset seizures. One interictal (2 hr) and one ictal (13.5 min) segment were analyzed with the SDA 111. The only seizure was first detected in the left ATN 162, where paroxysmal activity was present in all electrodes 130 in the left ATN 162, with a superficial (proximal) to deep (distal), high to low, amplitude gradient in referential montages. Thalamic ictal onset preceded scalp EEG onset by 2 seconds, and clinical onset by 3 seconds.

Subject 2: A 19-year-old man with right hemispheric, fronto-parietal, partial onset seizures. Two interictal (1.7 hr total) and 28 ictal segments (mean duration 124 s, SD: 86 s, range 60-341 s) were analyzed with the SDA 111. In 26/28 seizures, ictal activity was first detected in the right ATN 162, approximately 2 seconds prior to clinical onset and 20-25 seconds prior to the first rhythmic ictal scalp EEG changes; 2 events were undetected by the SDA 111.

Subject 3: A 34-year-old woman with symptomatic generalized epilepsy. Four interictal segments (1.2 hr total) and one segment (1.3 hr) containing one nocturnal generalized seizure were analyzed with the SDA 111. Ictal activity was first detected in the most superficial electrodes 130 of the electrode assembly 120 implanted in the left CMN 164, 0.5 seconds prior to clinical onset. Ictal scalp changes at clinical onset consisted of generalized high amplitude muscle and movement artifacts.

False positive (FP) seizure detections were rare with a seizure detection algorithm 111 applied to the interictal segments measured by the electrode assembly 120 surgically implanted in the ATN 162 or CMN 164 (0 FPs subject 1, 2 FPs subject 2, 0 FPs subject 3).

Typical medium to high amplitude surface negative scalp EEG interictal epileptiform discharges recorded over the temporal (subjects 1 and 2) or frontal (subjects 2 and 3) regions were recorded simultaneously in the thalamus but with opposite, positive polarity, representing subcortical volume conduction of the surface negative epileptiform discharges. Independently, in subjects 2 and 3, graphoelements compatible with interictal epileptiform discharges were recorded from electrodes 130 of the thalamic DBS electrode assemblies 120, the discharges usually limited to one or the other hemisphere, with no consistent relation to any visible scalp EEG waveform. Averaging a number of similar such discharges resulted in a low amplitude far field detectable at the scalp, however, individual discharges were typically unassociated with visible changes above background in the scalp EEG. Of interest, in subject 2, short-lasting (2-3 second) bursts of ictal-like activity not seen at the scalp EEG were recorded from the thalamus.

This example demonstrates the feasibility of subcortical (e.g., anterior or centro-median thalamic nuclei) seizure detection. The anatomical bases for the flow of afferences (physiological or ictal in nature) from certain cortical regions into the two thalamic nuclei targeted in this example (i.e., ATN 162 and CMN 164) are well known. The ATN 162 has: (a) direct reciprocal connections with the anterior limbic cortex situated rostrally and inferiorly to the corpus callosum, the cingulate gyms and the hippocampal gyms including the medial entorhinal cortex and the pre- and para-subiculum; and, (b) indirect connections with the hippocampal formation. The limbic cortical afferences to the ATN 162 are either bilateral or unilateral and originate mainly from layers V and VI. In human subjects, functional connectivity between mesial temporal structures and the ATN 162 or nuclei abutting this structure can be demonstrated using evoked responses. The CMN 164 has restricted reciprocal connections with the pre-motor, motor and supplementary motor areas in the frontal lobes.

Only in subject 1 did seizures originate from cortical regions projecting indirectly to the ATN 162. While accurate localization of epileptogenic tissue from the scalp EEG is fraught with pitfalls, the presumptive regions from where seizures in subject 2 emerged have no direct or indirect connections with the ATN 162, yet these partial seizures were detected from this nucleus (ipsilateral to the hemisphere of seizure origin). Subject 3's seizure was generalized, which explains why it was detected from the electrode assembly 120 implanted in CMN 164, and why it might also have been captured from the ATN 162 if an electrode assembly 120 had been implanted in the ATN 162.

So as to not destroy too many neurons during implantation, electrode assemblies 120 cannot be implanted into each of the “specific” and “non-specific” thalamic nuclei so as to enable detection from any/all epileptogenic regions. And thus a question of high practical relevance is: Are seizures that emerge from cortical areas without projections to either ATN 162 or CMN 164 detectable from either of these nuclei? The answer is a tentative yes, though the detection of seizures emerging from cortical regions without direct or indirect connections is perhaps more readily understood for CMN 164 than for ATN 162. The extensive intra-thalamic and inter-cortical connectivity of the reticular nucleus of the thalamus (RNT) may enable detection from the CMN 164 of seizures originating from regions without direct connection to it. Each RNT sector is connected to more than one thalamic nucleus and to more than one cortical area, serving as a “hub” where several functionally related cortical areas and thalamic nuclei interact. There is, however, one important and relevant exception: the ATN 162 is devoid of RNT afferences so detection of seizures from it, via the RNT, may be difficult or not possible. Explanation of detectability of seizures from the ATN 162 in subject 2 awaits further advances in knowledge of intra-cortical connections, human brain networks and the human “connectome.”

The occurrence of epileptiform discharges in thalamic recordings that are not visible at the scalp EEG, including short-lasting seizures, which may or may not show later propagation to the scalp EEG, suggests that the yield of paroxysmal epileptiform activity from thalamus may be higher and thus of greater utility than that of scalp EEG recordings.

This example supports the notion that detection of partial seizures from the CMN 164 or ATN 162 is beneficial and may serve as the basis for contingent detection and stimulation with devices, such as medical device 110. Subcortical seizure detection and contingent therapy delivery may benefit patients with pharmaco-resistant seizures, especially those with more than one “primary” epileptogenic region or in whom localization is difficult. Moreover, it may in certain cases obviate the need for precise localization of epileptogenic tissue.

It will be understood that implementations are not limited to the specific components disclosed herein, as virtually any components consistent with the intended operation of the various implementations may be utilized. Accordingly, for example, it should be understood that, while the drawing figures accompanying text show and describe particular embodiments and implementations, components may comprise any shape, size, style, type, model, version, class, grade, measurement, concentration, material, weight, quantity, and/or the like consistent with the intended operation of a methods and/or system implementations.

The concepts disclosed herein are not limited to the specific implementations shown herein. For example, it is specifically contemplated that the components included in particular implementations may be formed of any of many different types of materials or combinations that can readily be formed into shaped objects and that are consistent with the intended operation of the implementations. For example, the components may be formed of: rubbers (synthetic and/or natural) and/or other like materials; polymers and/or other like materials; plastics, and/or other like materials; composites and/or other like materials; metals and/or other like materials; alloys and/or other like materials; and/or any combination of the foregoing.

Furthermore, embodiments may be manufactured separately and then assembled together, or any or all of the components may be manufactured simultaneously and integrally joined with one another. Manufacture of these components separately or simultaneously, as understood by those of ordinary skill in the art, may involve extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled or removably coupled with one another in any manner, such as with adhesive, a weld, a fastener, any combination thereof, and/or the like for example, depending on, among other considerations, the particular material(s) forming the components.

In places where the description above refers to particular implementations, it should be readily apparent that a number of modifications may be made without departing from the spirit thereof and that these implementations may be applied to other implementations disclosed or undisclosed. The accompanying claims are intended to cover such modifications as would fall within the true spirit and scope of the disclosure set forth in this document. The presently disclosed implementations are, therefore, to be considered in all respects as illustrative and not restrictive, the scope of the disclosure being indicated by the appended claims rather than the foregoing description. All changes that come within the meaning of and range of equivalency of the claims are intended to be embraced therein.

What is claimed is:

1. A method for contingent thalamic stimulation triggered by automated seizure detection, the method comprising:
detecting electrical signals in a frequency band of 8-42 Hz from an anterior thalamic nucleus or a centro-median thalamic nucleus in a first hemisphere and/or a second hemisphere of a brain of a patient with one or more electrode assemblies surgically implanted at least partially within the anterior thalamic nuclei or the centro-median thalamic nuclei of the patient, and outside of a subthalamic nuclei of the patient;
transmitting the detected electrical signals to a medical device;
analyzing the detected electrical signals using a seizure detection algorithm; and
delivering electrical stimulation to the anterior thalamic nuclei or centro-median thalamic nuclei in the first hemisphere and/or the second hemisphere of the brain of the patient via the one or more electrode assemblies in response to a seizure detected by the medical device.

2. The method of claim 1, wherein analyzing the detected electrical signals using the seizure detection algorithm comprises determining whether a sudden increase in power of the electrical signals reaches a threshold value.

3. The method of claim 1, wherein the one or more electrode assemblies comprise between one to four electrodes having at least one of the electrodes surgically implanted within the anterior thalamic nuclei or centro-median thalamic nuclei.

4. The method of claim 3, further comprising selectively detecting electrical signals from any of the electrodes of the one or more electrode assemblies.

5. The method of claim 3, further comprising selectively determining any of the electrodes of the one or more electrode assemblies for delivering the electrical stimulation, the selective determination being based on at least one of: detected electrical signals and a therapeutic response to prior electrical stimulation.

6. The method of claim 1, further comprising detecting electrical signals from a cortical electrode array having a plurality of cortical electrodes.

7. The method of claim 6, wherein the cortical electrode array is implanted beneath the patient's skin and is operatively connected to the medical device, the medical device being configured to analyze electrical signals detected by the cortical electrodes using a cortical seizure detection algorithm.

8. A method for contingent thalamic stimulation triggered by automated seizure detection, the method comprising:
detecting electrical signals in a frequency band of 8-42 Hz from an anterior thalamic nucleus or a centro-median thalamic nucleus in a first hemisphere and/or a second hemisphere of a brain of a patient with one or more electrode assemblies surgically implanted at least partially within the anterior thalamic nuclei of the patient while bypassing a subthalamic nuclei of the patient;
transmitting the detected electrical signals to a medical device;
analyzing the detected electrical signals using a seizure detection algorithm; and
delivering electrical stimulation to the anterior thalamic nuclei in the first hemisphere and/or the second hemisphere of the brain of the patient via the one or more electrode assemblies in response to a seizure detected by the medical device.

9. The method of claim 8, wherein the one or more electrode assemblies comprise between one to four electrodes having at least one of the electrodes surgically implanted within the anterior thalamic nuclei.

10. The method of claim 9, further comprising selectively detecting electrical signals from any of the electrodes of the one or more electrode assemblies.

11. The method of claim 9, further comprising selectively determining any of the electrodes of the one or more electrode assemblies for delivering the electrical stimulation, the selective determination being based on at least one of: detected electrical signals and a therapeutic response to prior electrical stimulation.

12. The method of claim 8, further comprising detecting electrical signals from a cortical electrode array having a plurality of cortical electrodes.

13. The method of claim 12, wherein the cortical electrode array is implanted beneath the patient's skin and is operatively connected to the medical device, the medical device being configured to analyze electrical signals detected by the cortical electrodes using a cortical seizure detection algorithm.

14. A method for contingent thalamic stimulation triggered by automated seizure detection, the method comprising:
detecting electrical signals in a frequency band of 8-42 Hz from an anterior thalamic nucleus or a centro-median thalamic nucleus in a first hemisphere and/or a second hemisphere of a brain of a patient with one or more electrode assemblies surgically implanted at least partially within the centro-median thalamic nuclei of the patient, the one or more electrode assemblies also dodging a subthalamic nuclei of the patient;
transmitting the detected electrical signals to a medical device;
analyzing the detected electrical signals using a seizure detection algorithm; and
delivering electrical stimulation to the centro-median thalamic nuclei in the first hemisphere and/or the second hemisphere of the brain of the patient via the one or more electrode assemblies in response to a seizure detected by the medical device.

15. The method of claim 14, wherein the one or more electrode assemblies comprise between one to four electrodes having at least one of the electrodes surgically implanted within the centro-median thalamic nuclei.

16. The method of claim 15, further comprising selectively detecting electrical signals from any of the electrodes of the one or more electrode assemblies.

17. The method of claim 15, further comprising selectively determining any of the electrodes of the one or more electrode assemblies for delivering the electrical stimulation, the selective determination being based on at least one of: detected electrical signals and a therapeutic response to prior electrical stimulation.

18. The method of claim 14, further comprising detecting electrical signals from a cortical electrode array having a plurality of cortical electrodes.

19. The method of claim 18, wherein the cortical electrode array is implanted beneath the patient's skin and is operatively connected to the medical device, the medical device being configured to analyze electrical signals detected by the cortical electrodes using a cortical seizure detection algorithm.

20. The method of claim 14, wherein analyzing the detected electrical signals using the seizure detection algorithm comprises determining whether a sudden increase in power of the electrical signals reaches a threshold value.
